(19) United States
(12) Patent Application Publication
(10) Pub. No.: US 2020/0069848 A1
(43) Pub. Date: Mar. 5, 2020

(54) ANTIMICROBIAL INVASIVE SURGICAL DEVICES AND SYSTEMS

(71) Applicants: Peter C. Van Buskirk, Brookfield, CT (US); Maryam Golalikhani, Trumbull, CT (US); Jeffrey F. Roeder, Brookfield, CT (US)

(72) Inventors: Peter C. Van Buskirk, Brookfield, CT (US); Maryam Golalikhani, Trumbull, CT (US); Jeffrey F. Roeder, Brookfield, CT (US)

(21) Appl. No.: 16/278,092

(22) Filed: Feb. 16, 2019

Related U.S. Application Data
(60) Provisional application No. 62/632,312, filed on Feb. 19, 2018.

(51) Int. Cl. A61L 31/08; A61B 17/88; A61L 27/30; A61L 31/16; A61L 27/54

(52) U.S. Cl. CPC A61L 31/088; A61B 17/88; A61B 2017/00889; A61L 31/16; A61L 27/54; A61L 27/306

(57) ABSTRACT

Invasive surgical devices such as joint implants and surgical tools are provided with a coating of visible or near visible light stimulated TiO₂ based photocatalysts. The coatings may comprise one or more layers of different TiO₂ crystal phases and may incorporate metal nodules. The devices reduce the incidence of infection via antimicrobial and bactericidal surface properties. Related devices and methods for illumination, air purification, and packaging are disclosed that comprise a complete system for use of the devices in surgical procedures.

### CROSS REFERENCE TO RELATED APPLICATIONS

This application is a U.S. Utility application taking priority from U.S. Provisional application No. 62/632,312, “Antimicrobial invasive surgical devices and systems”, filed Feb. 19, 2018.

### FIELD OF THE INVENTION

The present invention relates to invasive, i.e. implanted, surgical devices that are placed in the body for short or long term periods and the provision of improved surfaces that impart antimicrobial benefits.

### BACKGROUND

Millions of joint replacements are performed every year. Periprosthetic joint infection (PJI) is a device-associated infection that poses a significant human and financial burden. Only a minority of joint arthroplasties become infected; however, these infections can cause significant morbidity, increase the risk of mortality, and contribute to a substantial proportion of health care expenditures. Treatment for PJIs usually involves multi-stage surgeries, which can lead to long hospital stays, delays in mobilization, pain, and large related costs per infection.

The majority of PJIs are the result of microorganisms introduced to the implant surface at the time of surgery through direct contact or aerosolized contamination. Planktonic microorganisms colonize the surface of the implant, and biofilm development begins. Biofilms are organized structures with numerous microorganisms surrounded by a self-produced matrix. Early biofilms are relatively unstable and still susceptible to host defense and antimicrobial agents. During biofilm maturation, a high density of microorganisms will form and provide the physiologic condition for microbial communication systems, i.e., quorum-sensing. Quorum-sensing regulates the production and release of various virulence factors protecting the biofilm from destruction. When organized in biofilms, microbes can be up to 1000-fold more resistant to antimicrobial drugs compared to planktonic microbes.

Other devices can cause infections that are ancillary to PR For example, contaminated surgical instruments can introduce pathogens to the surgical site. External fixation pins that penetrate the skin have high rates of infection.

The formation of biofilms at the surface of medical devices is governed by the interactions among the device, the host, and the bacteria. Modification of the device surface provides a significant opportunity for preventing biofilm formation. To enhance the clinical outcome, the orthopedic implant material should prevent biofilm formation while, at the same time, possess other properties, e.g, osteogenic properties to lead to osseointegration. Osseointegration promotes new bone formation on and around the implant, and is an important characteristic of the implant surface for a successful implantation of a variety of devices, e.g, joint implants, dental implants, etc. The surface characteristics of the implant highly affect the osseointegration. These characteristics include surface chemistry, topography, wettability, charge, surface energy, crystal structure and crystallinity, and roughness. Surface chemistry refers to the oxide present on Ti or other metal based alloys. In the case of Ti based alloys, the surface is a native layer of TiO₂ in the range of 3-10 nm. TiO₂ has good osseointegrative properties and low cytotoxicity. Surface topography has several scales. Macroscopic topography in the 0.1-3 mm range imparts a capability for strong bonding to bone in certain regions of an implanted device. Topography in the microscale (1-10 micron) and nanoscale range (1-500 nm) also imparts antimicrobial and osseointegrative properties. Hydrophilic surfaces promote bone growth and anti-adhesion characteristics with respect to bacteria and biofilm formation. Charge characteristics affect initial bone-tissue interaction in the very early stages of osseointegration (seconds). Therefore it is important that any coating have similar or improved charge characteristics relative to the surface of the bulk device being implanted. Surface energy is related to wettability. Hydrophilic surfaces have higher surface energies and a preference for water molecules compared to other molecules. As described herein, there are varying degrees of crystallinity and crystal phases that may be present on an invasive device surface. For the case of Ti based alloys, the native oxide is often amorphous. For optimal surface energy and wettability, crystalline phases are may be used to tune the surface energy and wettability. The anatase phase, alone or in combination with other crystalline phases including rutile and brookite are useful in this regard. Roughness corresponds to the surface topographies, and may be expressed as the highest roughness observed (Zmax) or the root mean squared average roughness (RMS roughness). Orthopedic surgery implants are commonly made of titanium (Ti) and its Ti-6Al-4V alloy, both of which are bioinert and corrosion resistant, have a low Young's modulus, and most importantly are osteogenic. To achieve antimicrobial properties, researchers have investigated various strategies that focus on generating non-adhesive and/or bactericidal surfaces that can potentially prevent colonization or interrupt biofilm maturation, such as adding metal ions to implant materials and permanently binding antibiotics to implant surfaces. Drawbacks to these approaches include regulatory burdens, molecule instabilities, and local bacterial resistance.

Much current work is focused on surface nanostructures of the base implant material that interfere with bacterial adhesion and proliferation, some of which mimic biological structures, and are on the order of 10s to 100s of nm in scale.

The surface of Ti-containing implant devices is TiO₂, a natively formed oxide that can be photocatalytic. Reactive oxygen species (ROS) created during photocatalysis are broadly antimicrobial. However, a limitation of native TiO₂ implant surfaces is the need for high intensities of ultraviolet (UV)-A or UV-B light to activate the photocatalytic effect given the relatively low photocatalytic activity of amorphous native surfaces, as well as typical formation of the less photoactive phases of TiO₂ such as rutile or brookite. UV light is hazardous to humans and cannot be used continuously at high intensities for disinfecting medical devices in the presence of health care personnel and patients. This limitation has significantly restricted the adoption of photocatalytic TiO₂ in this application. Finding a way to exploit photocatalytic effects at the time of surgery opens a window of opportunity for preventing bacterial contamination of implants.

Antimicrobial properties may arise from more than one property of a surface. Surfaces to which bacterial are less likely to adhere are antimicrobial because the bacteria cannot form colonies that can further organize into biofilms. Surfaces can also be bactericidal meaning that they have properties that kill bacteria. The photocatalytic surfaces described in this patent are bactericidal because the ROS attack the cell calls of the bacteria and destroy their ability to survive or replicate. They may also inhibit adherence of bacteria. Both anti-adhesion and bactericidal actions are antimicrobial.

Accordingly, it would be a significantly advantageous improvement to have a photocatalytic surface on invasive surgical devices that produces an antimicrobial effect, is non toxic, and is compatible with other intentional surface nanostructures along with appropriate systems for illumination, and other ancillary materials and systems, including those for maintaining sterility, activating the antimicrobial surface, potentiating its antimicrobial properties, and facilitating its remaining active while exposed to the operating room environment.

Another example of invasive medical devices which are prone to infection are external fixations. They are a commonly used technique of bone fracture fixation among orthopedic surgeons after major trauma. Pins are used to provide alignment during healing and may also be supplemented with wires. These devices penetrate the skin. Infection is one of the most common and most important complications of external fixation. Infection usually occurs around pin and wire sites where they penetrate the skin.

Systemic antibiotics are usually used to prevent or cure this type of infections. However; the effectiveness of antibiotics is limited because they may not penetrate to the infection site and some pathogens have strong resistance to antibiotics, particularly on surfaces in the form of a biofilm, as discussed previously.

Modifying the external fixation surfaces for antimicrobial effect would be of a great advantage. As described below, the combination of modified surfaces with an illumination system can be used to keep the surface of the device active and prevent infection.

### SUMMARY OF THE INVENTION

The present invention relates to an invasive surgical device with a modified surface (“invasive device modified surface”) that is antimicrobial, and comprised in part by a highly conformal antimicrobial coating. In a related aspect of the invention, the coating materials and physical integrity allow normal use of the invasive surgical device, including low cytotoxicity, and excellent osseointegration properties if required for the device.

In one aspect of the invention, the antimicrobial properties of the coating are activated, i.e. induced, by incidence of light. The light source is a LED which comprise visible wavelengths (greater than 400 nm) and may be extended to UV-A and UV-B wavelengths. In a related aspect, the thickness and materials design of the coating has been engineered to enhance the antimicrobial photo-response of the surface to the target wavelength.

In another aspect, the invention relates to an invasive device modified surface that is comprised of one or more nanometer scale layers, formed in a series of coating deposition and heat treating steps that are employed to form the invasive device modified surface.

In a related aspect of the invention, nano topographic features can be developed by these additive and heat treating process steps, possibly but not necessarily combined with subtractive processing steps.

In another aspect, the invention relates to the specific attributes of the modified surface so formed.

The invention also includes non-invasive surfaces in proximity to invasive surfaces, such as tables, trays, and instrumentation panels and other instruments.

In yet another aspect, the invention relates to packaging and activation means to stimulate the antimicrobial surface via light. In a further aspect, the invention relates to the illumination sources used to stimulate the antimicrobial surface.

In still another aspect, the invention relates to use with liquid or mist agents that increase photocatalytic activity.

### BRIEF DESCRIPTION OF THE DRAWINGS

FIG. 1 shows an orthopedic implant with modified surface.

Figure [00022] shows a modified surface, with a two-layer film structure that is conformal on a surface with nano or micro topographic features.

FIG. 3 shows modified surface layer structure, incorporating anatase/native oxide on chemically pure titanium.

FIG. 4 shows modified surface layer structure, incorporating rutile/anatase on a Ti-6Al-4V substrate.

FIG. 5 shows modified surface layer structure, incorporating rutile/Pt nano-islands/anatase on 316L stainless steel.

FIG. 6 shows modified surface layer structure, incorporating rutile nano-islands in anatase film matrix.

FIG. 7 shows modified surface layer structure, incorporating a TiO₂—CeO₂ anatase layer.

FIG. 8 shows modified surface layer structure, incorporating a CeO₂/anatase bilayer.

FIG. 9 shows modified surface layer structure, incorporating a CeO₂— TiO₂ rutile/anatase bilayer.

FIG. 10 shows a flow chart for fabrication of multilayer rutile-anatase with incorporation of Pt nanostructures.

FIG. 11 shows a flow chart for fabrication of rutile nano-islands in anatase.

FIG. 12 shows a flow chart for fabrication of porous structures to contain antimicrobial materials.

FIG. 13 shows a flow chart for fabrication of topographic structures.

FIG. 14 shows an x-ray diffraction pattern for an annealed Ti-6Al-4V substrate surface showing rutile crystal phase.

FIG. 15 shows an x-ray diffraction pattern for a deposited amorphous film and the same film converted to rutile crystal phase post deposition.

FIG. 16 shows an x-ray diffraction pattern for an anatase film on a rutile crystal phase under-layer.

FIG. 17 shows an x-ray diffraction pattern for an anatase film (as-deposited) on a Ti-6Al-4V substrate.

FIG. 18 shows an optical model of a TiO₂ film on a titanium substrate a) Spectral reflectance, b) E-field distribution for illumination at 405 nm.

FIG. 19 shows an optical model of a TiO₂/Al₂O₃ bilayer on a titanium substrate a) Spectral reflectance, b) E-field distribution for illumination at 405 nm.

FIG. 20 shows an optical model of a TiO₂/rutile/Al₂O₃ trilayer on a titanium substrate a) Spectral reflectance, b) E-field distribution for illumination at 405 nm.

FIG. 21 shows antimicrobial data for an anatase film on a silicon substrate irradiated at 365 nm.

FIG. 22 shows a contact angle image before and after UV irradiation.

FIG. 23 shows a schematic of an orthopedic invasive device in transparent packaging during illumination prior to its use.

FIG. 24 shows a schematic of a photocatalytic coating over wear resistant particles adhered to the substrate.

### DETAILED DESCRIPTION OF THE INVENTION, AND PREFERRED EMBODIMENTS THEREOF

Invasive surgical devices come in contact to tissue within the body. Contact may occur for short times or for extended periods. Examples of invasive devices that come in contact inside the body for short times include various surgical instruments, including forceps, needles, retractors, clamps, and the like. Short exposures may vary from seconds to hours, depending on the device and procedure. Examples of invasive devices that remain in the body for extended periods of time (years) include orthopedic implants, plates, screws, and the like. Some invasive devices have contact within the body for intermediate periods of time (days-weeks). Devices in this category include fixating pins that penetrate the skin, temporary implants and trauma related devices.

Each device has a certain set of key requirements and common requirements that may be more or less important. For example, osseointegration is very important for implants and screws, but unimportant for forceps and external fixating pins. The degree of cytotoxicity is important for all devices, but most important for intermediate and long-term devices. Surgical instruments like forceps require a highly wear resistant surface. Antimicrobial properties are important for any device of an invasive nature, because each contact of within the body with a foreign object that could be contaminated carries with it the risk of infection.

Invasive devices may have different chemical compositions. Many long term invasive devices are titanium based. The alloy Ti-6Al-4V is a common alpha-beta titanium alloy with favorable mechanical properties compatible loadings encountered with human bone. The stem and neck of a hip implant are commonly Ti alloys. Commercially pure titanium (CP—Ti) is also commonly used. A number of other Ti alloys are in use or being considered for use, including Ti-6Al-7Nb, Ti-5Al-0.5B, Ti-16Nb-13Ta-4Mo, Ti-12Mo-6Zr-2Fe, Ti-15Mo-5Zr-3Al, Ti-29Nb-13Ta-4.6Zr. Ti-13Nb-13Zr, Ti-29Nb-13Ta-4Mo, Ti-29Nb-13Ta-4.6Sn, Ti-29Nb-13Ta-6Sn, Ti-29Nb-13Ta-2Sn, Ti-29Nb-13Ta-4.5Zr, and Ti-29Nb-13Ta-7.1Zr. The femoral head of an implant may be Cr—Co. Surgical instruments are often stainless steel alloys, including alloy 316 and 400 series alloys. Fixating pins may be stainless steel or titanium based. Ceramics may also be used, including Zr stabilized alumina and yttria stabilized zirconia, for example as femoral heads. Finally, polymeric materials find use in invasive applications, particularly poly-ether-ether-ketone (PEEK). It would be highly advantageous to have antimicrobial coatings that are compatible with each of these materials.

Surface morphologies of certain implant devices may be varied, for example to promote osseointegration. The scale of the morphology (e.g., roughness, undulations, and 3-dimensional features) may be on the order of millimeters, micrometers, and nanometers. Nanometer scale features have some advantages with respect to providing naturally antimicrobial behavior due to geometrically related interactions with pathogens. It is an object of the present invention to retain these useful features via a highly conformal coating. The coating scheme can also be used to produce nanoscale features to further improve antimicrobial and osseointegration.

The present invention relates to an invasive surgical device with a highly conformal antimicrobial coating and systems to support maintaining a sterile surface while exposed to the operating room ambient environment. Conformality is defined as the ratio (in percent) of the thickness at the thinnest place in the coating divided by the thickness at the thickest place in the coating. Invasive surgical devices include, but are not limited to, implants and surgical tools. Implants include dental stems, orthoscopic implants, plates, screws, and the like. Surgical tools include forceps, retractors, drills, saws, needles and the like. Environmental microbes, e.g., planktonic bacteria, etc. may contaminate otherwise sterile surfaces while they are exposed during use or handling in the operating theater. These microbes may then multiply within the body and lead to biofilm formation and/or PJI.

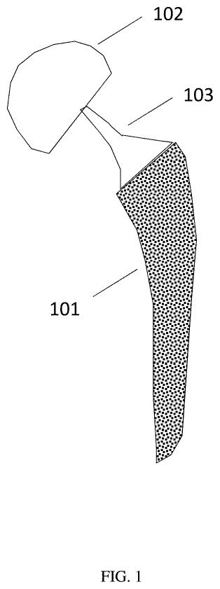

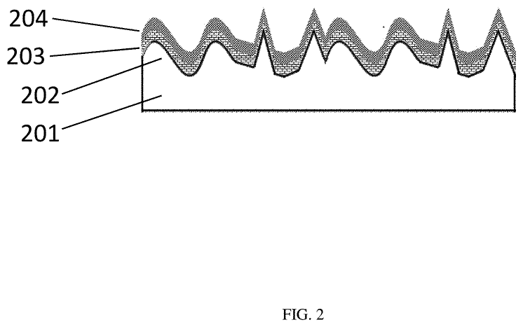

The surfaces of implants have different topographic features depending on the location. For example, referring to FIG. 1, the stem 101 is often a porous metal that has a large surface area that promotes a strong bond with bone tissue. Such features are on the millimeter to micrometer scale. Some features are fibrous or filamentary and separated by open regions on the same scale as the feature itself. Other surfaces such as the ball 102 may be smooth, and are load bearing surfaces that may be prone to wear. Still other surface features may have nanostructures (i.e., features at the 10's of nm length scale) that are intentionally provided to impart some level of functionality to the surface, including antimicrobial properties by virtue of providing a surface that is a poor host to microbes. The coating of the present invention provides a uniform conformal coating that replicates these features at all of the relevant dimensional scale lengths and further, would augment any intrinsic antimicrobial properties with active antimicrobial properties.

In this application, the term coating and layer may be used interchangeably. A coating may comprise one or more layers.

The specific attributes of the photocatalytic coating are the ability to provide bactericidal, viricidal, and sporicidal properties while it is illuminated with light of wavelengths >360 nm, preferably 380 nm, and more preferably 400 nm. The coating has a density of ≥90% and preferably ≥95%. The thickness of the coating is ≤50 nm, and preferably ≤30 nm. Conformality may range from 75-100%, more preferably from 85-100% and most preferably from 90-100%. Certain dopants may be used to shift the photocatalytic response into the UVA and visible light ranges. The CeO₂ content may be between 0.1 and 5% (all percentages expressed herein are in atomic %) for a film in which the CeO₂ is distributed throughout the thickness of the film. The CeO₂ may be incorporated as discrete layers into the film. There may be one or more CeO₂ layers in the TiO₂ film. Spacings between layers may be from 1 to 10 nm. Other nanoparticles or phases may be disposed within the film, e.g., plasmonic particles of metals like Ag or other elements that act as charge injection regions or have wear reducing properties (e.g., Al₂O₃, SiC, BN). In all cases any second phase region is effectively locked into the film, in contrast to free nanoparticles that may migrate within the body. The photocatalytic surfaces of the subject antimicrobial devices require illumination at appropriate wavelengths to induce these desirable effects, and there are a variety of methods to deliver photocatalytic illumination to the subject surfaces. The photocatalytic illumination wavelengths are preferably in the visible spectral region, i.e. 400-700 nm wavelength range, especially in the 400-460 nm range which is generally more efficacious for creating reactive oxygen species (ROS) by means of the photocatalytic effect (i.e. the interaction with holes and electrons with ambient species like O₂ and H₂O). In certain embodiments, especially in dedicated pre-packaging orthopedic implant or surgical instruments, UVA illumination (360-400 nm wavelength) sources may also be employed for in-vivo emission sources, or for fiber or other geometric means to deliver photocatalytic illumination to the subject surfaces. In those cases, the use of UV radiation would not pose a threat to operating room personnel.

Photocatalytic illumination may be preferably provided by light emitting diodes (LEDs). The most efficient of these sources in the 360-460 nm range employ InGaN LED epitaxial emitting structures. These illumination sources may be combined with other illumination sources that are implemented for general illumination (i.e. room ambient lighting, operating table and surgical cavity task lighting), or for the purpose of photodynamic therapy, or for laser surgery, or for other optical functions in the operating theater. Combination of such multifunction illumination sources may be accomplished in hybrid LED module implementation that combine different types of emission sources at the board level, or via other geometric means that combine dissimilar spectral components via geometric means. Dissimilar illumination wavelengths may be generated entirely by LEDs, or by a combination of different light sources including laser diodes, HID lamps, gas discharge sources (e.g. xenon discharge), solid state lasers, up or down converted emitters, etc.

In one embodiment, operating theater general illumination is provided by LED luminaires that incorporate broadband visible illumination and dedicated short wavelength visible illumination. In a preferred embodiment, the short wavelength photocatalytic illumination is predominantly in the 405-420 nm wavelength range. The photocatalytic antimicrobial surfaces of invasive devices, either implants or surgical instruments may in that case be optimized for that illumination wavelength range. 405 nm radiation is also known to directly dissociate kill bacteria by direct interaction with matter inside a bacteria. In those cases the bactericidal effects would be complementary.

In another embodiment, photocatalytic illumination may be provided as an element of dedicated task lighting that is directed specifically to an operating table or into the surgical cavity. Such illumination may be broadband (for example as provide by a Xe or HID lamp), or by appropriate LED sources as described above. Specialized luminaire sources may direct the illumination into the surgical cavity via conventional spotlights, fiber optic or other types of light guides. These light guides may also include illumination for other surgical functions, or general illumination. They may also be attachable to the surgeon's headgear. Other examples of dedicated lighting include lighting modules placed in proximity to dermal penetration of fixating pins. These modules may provide light from point sources that form an annular illumination pattern at the juncture of the pin and epidermis.

Dedicated photocatalytic task lighting may be provided on the operating table by integration into a laminar flow system that provides bactericidal, virucidal and sporicidal treatment of the ambient air environment where surgical devices are removed form their packaging, and on or near the surgical cavity. An illumination device may also provide ancillary air purification, for example, using a photocatalyst to produce ROS that purify an air stream that flows through the device and sweeps the surface of invasive instruments. This may be embodied as an illumination source proximate to the invasive device combined with an air purification device that provides a down draft purified air curtain. In this way, the photoactive surface of the device is illuminated in addition to being protected from contaminated air in the ambient in the operating room. The illumination device may provide light in the 365-410 nm range, and preferably from 405-410 nm.

In another embodiment the light guide output termination may be temporarily attached to one aspect of the invasive surgical device, so as to provide continuous illumination. In that manner a light guide illumination providing function may function post operatively at surfaces of an implanted device, after the surgical incision is closed. In that case the light guide would protrude through the closed incision. Photocatalytic illumination light guide sources of that type may be combined with fluid introducing means, where the fluid is an antibiotic, a photodynamic therapy dye, or other medication containing media. The fiber optic illumination light guide may also be combined with a drain tube.

In another embodiment, photocatalytic illumination may be provided in vivo, after a surgical implant operation, by implantable LED light sources that are packaged for long term operation in the body. These sources may incorporate micro-LEDs or normal LEDs with die size on the order of 1 mm, packaged with a power source such as a battery, or an RF inductively or other remote energy transmitting and LED control means. Such in vivo implantable devices may be positioned near an implanted surface, or in the interior of an implanted surface that is optically transmissive at photocatalytic illumination wavelengths. Transmissive implant materials include zirconia containing ceramics and PEEK or similar polymers.

In another embodiment photocatalytic illumination may be provided in vivo by direct infrared illumination of infrared photocatalytic activated surfaces, through body tissue that typically has penetration depths of millimeters to centimeters for near infrared spectral.

Several chemical additives that may be provided to the subject photocatalytic antimicrobial surfaces to transitively increase photocatalytic and hence antimicrobial efficacy. One route to enhance photocatalytic effects is via addition of peroxide containing aqueous solutions, especially at concentrations less than 0.05 molar. Another route includes Fenton reactions, which utilize iron or other ions provided at a surface. Photocatalytic enhancing additives could therefore incorporate individual or combinations of peroxides, sources of iron or related ions. Photodynamic therapy utilizes dyes as photoreceptor media to generate reactive oxygen species, analogous to photocatalytic surface effects. Liquid enhancing media could accordingly incorporate such photodynamic dyes as components of the peroxide and/or Fenton reaction liquid media.

Such liquid media would preferably be provided to the antimicrobial implant surface ex vivo or in the surgical cavity, as a nebulized suspension or mist of micro droplets, with sizes appropriate to penetrate porous implant surface topographies. Such nebulized media could be supplied by the air cleaning or photocatalytic illumination providing devices near the operating table. The nebulized vapor may also be provided as part of and within the smart packaging of the subject invention, for the implant or surgical instruments. In that case it may be actuated and delivered to the surface on demand at some time before opening the package, in order to enhance the antimicrobial efficacy.

Such liquid media could also be provided to either interior or exterior surfaces of subject implant devices, during surgery or post-surgery (i.e. in vivo). Provision of such liquid enhancing media in vivo may be provided via a dedicated tube or as part of a laparoscopic device that combines photocatalytic illumination, general illumination for imaging, optical or mechanical surgical functions, or other liquid transfer functions.

The invention includes dedicated packaging methods as a means to enabling or improving the efficacy of the subject photocatalytic surfaces. In one embodiment, a package directly provides photocatalytic illumination from optical sources that are integrated in the package. Illumination from single or multiple sources is directed to the hardware surface via reflective and/or transmissive elements built into the package. The illumination is directed at all or most of the subject surfaces. In order to achieve adequate illumination within the porous topography, a range of incidence angles may be engineered for the illumination, using scattering exit surfaces of a waveguide structure for example. A related approach is to use multiple micrometer or sub-micrometer scale light guides to deliver illumination near a surface. Another aspect for achieving adequate illumination is to provide excess intensity at each surface, such that small fractions of the incident intensity inside pores will be sufficient to achieve antimicrobial efficacy. Another embodiment utilizes and external illumination source, which is provided to an input port, multiple input ports or other transparent surfaces of the subject packaging.

In another embodiment the smart packaging provides enhancing photocatalytic or optically stimulated media to the device surface prior to removal and use of the device in a surgical operation. These fluid media may include nebulized peroxide, Fenton reactants, photodynamic enabling dyes or other media.

Control of the illumination and enhancing media delivery functions may be externally actuated and/or controlled by remote means, including wireless communication such as Bluetooth, WiFi, or RFID technologies that are well known. For example, a liquid reservoir and a battery powered nebulizer may be integrated in the packaging, in or near the optical illumination optics, and these subsystems may be remotely actuated at some time prior to use in the operating room. The smart packaging functions may include tracking device history and logistics, including GPS, and measurement of optical illumination time and intensity at or near representative device surfaces. Control of the packaging may be integrated into operating room or hospital wide control system to track and control critical equipment, materials, patients, environmental or other factors.

### EXAMPLES

The subject invention may be embodied in the forgoing examples that are by no means restrictive, but intended to illustrate the invention.

The basic innovation is comprised of an invasive surgical device, or related devices, principally those used in surgery, with a modified surface that possesses antimicrobial properties that are stimulated by incident illumination. This resulting antimicrobial property may be completely caused, or caused in part by the photocatalytic effect. FIG. 1 shows an orthopedic implant used in hip reconstruction, with modified roughened surface on the stem 101 where it osseointegrates with the bone.

In some embodiments a second antimicrobial mechanism may be employed in addition to the photocatalytically actuated microbial surface. One case is the use of nano or micrometer scale topographic features that are known to reduce the growth of undesirable biofilm on or near joint implant surfaces in the body, e.g. bacterial infections. In the case of complementary topographic and photocatalytic antimicrobial surface technologies, it is highly desirable for the photocatalytic layer or layers to be conformally formed on the underlying topography. FIG. 2 shows a modified surface, with a two-layer film structure that is conformal on a surface with nano or micro topographic features. In that case, 201 is the titanium alloy substrate, 202 is the preexisting topography, 203 is a first conformal layer of TiO₂, and 204 is a second conformal layer with photocatalytic properties. It is evident that the final topography will be nearly identical to the underlying topography, while a desirable photocatalytic properties superimposed, thus providing two antimicrobial technologies. Conformality of the coating may be between 75-100%, preferably 85-100%, most preferably 90-100%. Conformality of the first layer may be between 75-100%, preferably 85-100%, most preferably 90-100%. Conformality of the second layer may be between 75-100%, preferably 85-100%, most preferably 90-100%. The thickness of the first layer may be from 10-100 nm, preferably from 10-30 nm. The thickness of the first layer may be from 10-100 nm, preferably from 10-30 nm.

Titanium oxide is a photocatalytic material and the anatase crystal phase has the highest photocatalytic activity, partly due to the high density of hydroxyl radicals that certain crystal faces in anatase may possess. The density of hydroxyl radicals also depends on the photocatalytic illumination wavelength, and the interaction of light with that wavelength, in terms of optical absorbance of the material at the illumination wavelength.

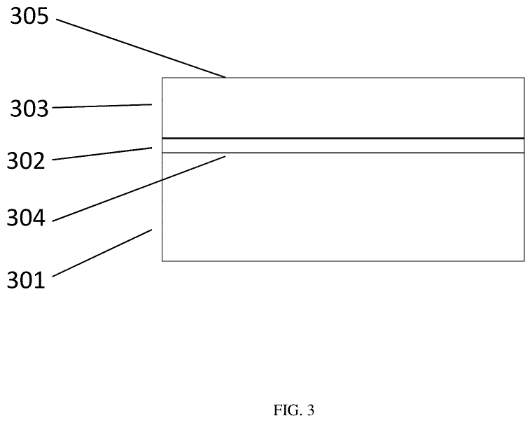

A preferred embodiment of the subject invention is the presence of anatase titanium oxide (“anatase”) on the outer surface of the modified surface. FIG. 3 shows modified surface layer structure, incorporating a two-layer coating on chemically pure titanium or titanium alloy 301, comprised of a native titanium oxide 302 and anatase 303. Anatase layer thickness is in the range of 10-100 nm, more preferably 20-45 nm. Such thin layers of anatase may contain very small crystallites in the range of from ⅕ the film thickness to 5 times the film thickness. For larger crystallite sizes the surface roughness of the top layer 305 may be larger than the surface roughness of the substrate topography 304, i.e. the film deposition and crystallization may introduce its own topography. The layer structure shown indicates a planar substrate surface 304, although this multilayer film system may be formed on topological surfaces such as in FIG. 2.

Conformality of the coating may be between 75-100%, preferably 85-100%, most preferably 90-100%. Conformality of the first layer (native oxide) may be between 10-100%, preferably 50-100%, most preferably 90-100%. Conformality of the second layer may be between 75-100%, preferably 85-100%, most preferably 90-100%.

This film formation topography will have its own characteristic average surface roughness and values of roughness within the distribution of roughnesses. The final combined topography may result as the superposition of substrate topography, first layer topography, second layer topography 305. This combined topography may be engineered to increase antimicrobial properties, including antimicrobial effects hours or days after the device has been implanted.

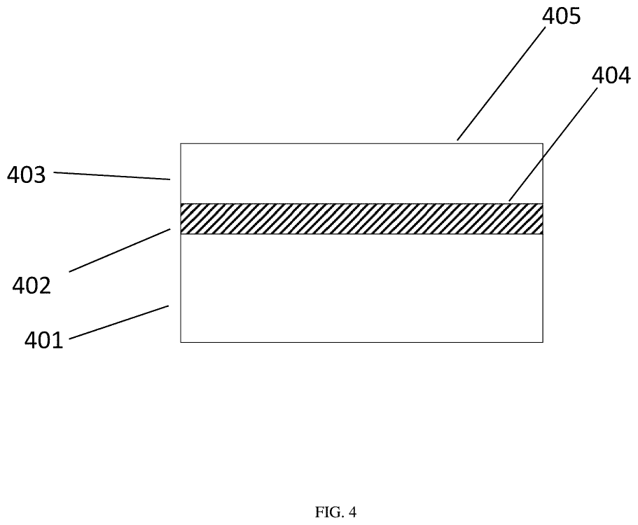

The titanium surface typically has a somewhat thin native titanium oxide on its surface that is amorphous, typically in the range of 4-12 nm thickness, depending on its processing and storage history. In the case of intentional surface modifications prior or after subsequent film deposition, the native oxide may be thought of as a thermal oxide, and its thickness may increase, or its crystallinity may convert to anatase, rutile or brookite, or a combination thereof. A preferred embodiment of the modified surface layer structure is shown in FIG. 4, incorporating a 2 layer structure comprised of titanium oxide with rutile crystal phase (“rutile”) 402 adjacent to the Ti-6Al-4V substrate 401, and a top layer of anatase 403. This structure has several desirable characteristics for improving the photo-response of the modified surface. Rutile is known to have a smaller optical band gap than anatase, and hence it has higher optical absorption near the band gap, at wavelengths where anatase has lower absorption. While not being bound by theory, the rutile layer can serve to absorb photons, and the resulting charge separation into TiO₂ may be injected into the anatase layer to create reactive oxygen species and the photoelectric effect at the anatase outer surface 404. The rutile-anatase interface 405 may also in itself contribute to charge separation in the anatase layer. The thickness of the anatase layer 403 is in the range of 10 nm-100 nm, and preferably in the range of 15-50 nm. The thickness of the rutile layer 402 is in the range of 10-80 nm, and more preferably in the range of 10-30 nm. Examples provided below will describe how to improve optical coupling into the higher absorption layers such as rutile 402 or other layers, and how to form such anatase 403 or rutile 402 layers using deposition and thermal annealing processes. Conformality of the coating (anatase layer) may be between 75-100%, preferably 85-100%, most preferably 90-100%.

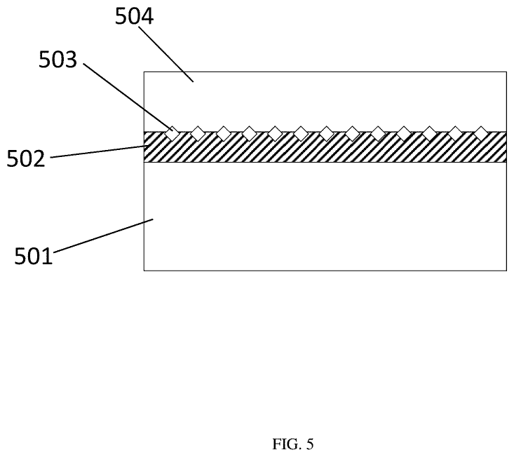

The modified surface layer structure in the previous example may be extended to incorporate platinum islands. In this case the platinum islands enhance the photocatalytic activity of the modified surface by acting as a reservoir of electrons, thereby retarding recombination of electrons and electron holes. FIG. 5 shows this modified surface layer structure, incorporating rutile layer 502 on a stainless steel of Ti alloy substrate 501. Platinum nano-islands 503, typically 2-20 nm size, are formed on the rutile top surface, followed by formation of an anatase layer 504 as the outer surface. Other platinum group metals (PGMs) such as palladium, iridium and rhodium are also suitable for this function. The overlying anatase layer 504 is less than 5-20 nm thickness, these Pt nano-islands may also provide an antimicrobial effect that is independent of the photocatalytic effect. Methods for forming such structures are provided in the examples below.

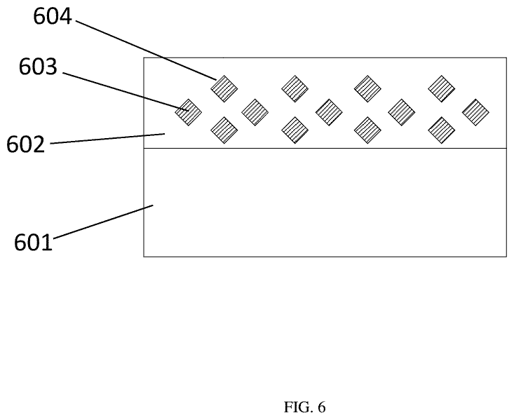

Another example utilizing rutile and anatase as components of a layer structure is shown in FIG. 6. This modified surface layer structure incorporates rutile nano-islands 603 in an anatase film matrix 602, formed on a Ti-6Al-4V alloy substrate 601. Such rutile crystallites are in the 3-20 nm size range, and serve as distributed absorbers and charge injectors, with enhanced rutile-anatase interface area 604. The total thickness of the coating may be from 10-100 nm.

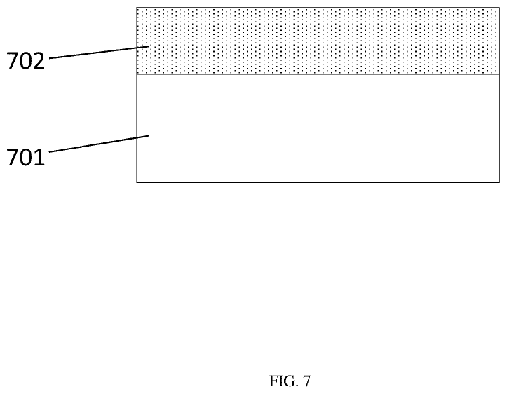

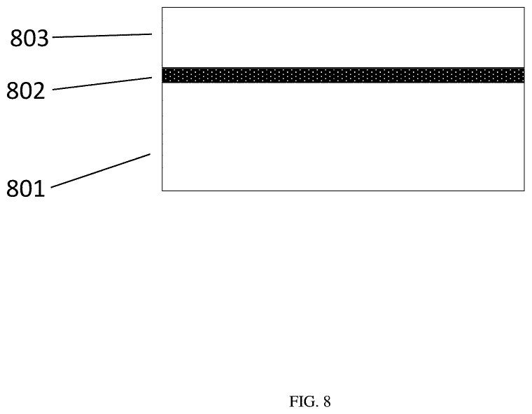

CeO₂ may also be incorporated in an anatase layer, as shown in FIG. 7. In this case a Ce is incorporated in the anatase lattice on Ti sites, providing a macroscopically homogeneous TiO₂—CeO₂ anatase layer (“CeO₂ containing anatase”) 702 on a chemically pure (CP) titanium or titanium alloy substrate 701. The CeO₂ content may be between 0.1 and 5% for a film in which the CeO₂ is distributed throughout the thickness of the film. Another embodiment is provided in FIG. 8, showing a bilayer modified surface layer structure, incorporating a discrete CeO₂ layer 802 with an anatase layer 803, on a 316L stainless steel substrate 801. The layer thickness of the CeO₂/TiO₂ may be between 10-50 nm. The thickness of the CeO₂ may be between 0.2-2 nm.

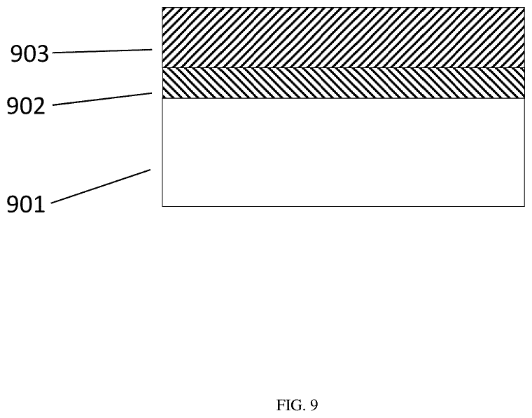

CeO₂ may also be incorporated into the titania rutile crystal lattice (“CeO₂ containing rutile”), and that thin film material may be incorporated into layer structures via methods provided herein. FIG. 9 shows modified surface layer structure, incorporating a CeO₂ rutile 902, with an anatase over layer 903, synthesized on a CP titanium or Ti alloy substrate 901. The overall thickness of the TiO₂—CeO₂ composite may be 10-50 nm. There may be one or more discrete CeO₂ layers in the TiO₂ film. Spacings between layers may be from 1-10 nm. Thicknesses of the CeO₂ layers may from 0.3-2 nm.

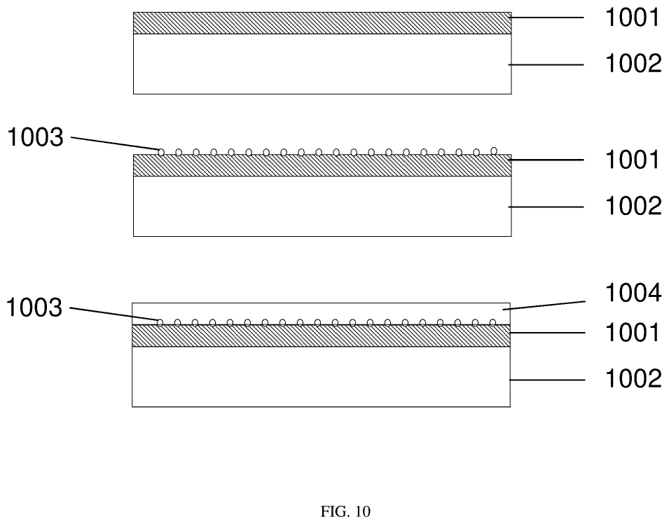

In another example, platinum or other noble metals may be embedded into an anatase layer to provide a reservoir of electrons and optically enhancing structures that excite plasmons. FIG. 10 shows a fabrication sequence of multilayer rutile-anatase with incorporation of Pt nanostructures. In this embodiment, a rutile layer 1001 may be formed on a substrate 1002 by annealing a Ti containing substrate under the appropriate conditions or by depositing a rutile film or an amorphous TiO₂ film that is converted to rutile by post-annealing. The rutile may have topology. Pt or other noble metal (Ir, Pd, Rh) nodules 1003 may be deposited by ALD or by a solution treatment. Suitable ALD precursors for Pt include Pt(Cp-Me)Me₃, where Cp=cyclopentadienyl and Me=methyl. Oxygen or a reducing gas such as hydrogen may be used as the reactant. In the case of a solution treatment, PtCl₂, PtBr₂ or PtI₂ may be dissolved in water and dispersed on the substrate. The substrate is then calcined in air or oxygen. Other Pt salts that may be used include Pt(NH₃)₂Cl₄, H₂Pt(OH)₆, and Pt(NH₃)₂Cl₂. The Pt nodule size may range from 1-20 nm and the inter-nodule spacing may vary from 1-1000 nm. Preferably, the inter-nodule spacing is 5-100 nm. An anatase layer 1004 is then deposited over the nodules. The thickness of the anatase layer may be from 5-100 nm, more preferably from 10-30 nm. The anatase layer 1004 may be deposited by ALD as described elsewhere herein. The TiO₂ layer may also be deposited in the amorphous state and then annealed to form anatase. In another embodiment, an anatase layer may be deposited first on the rutile layer, followed by the Pt nodules and then a second anatase layer to contain the nodules within the anatase layer. In yet another embodiment, the rutile layer is omitted.

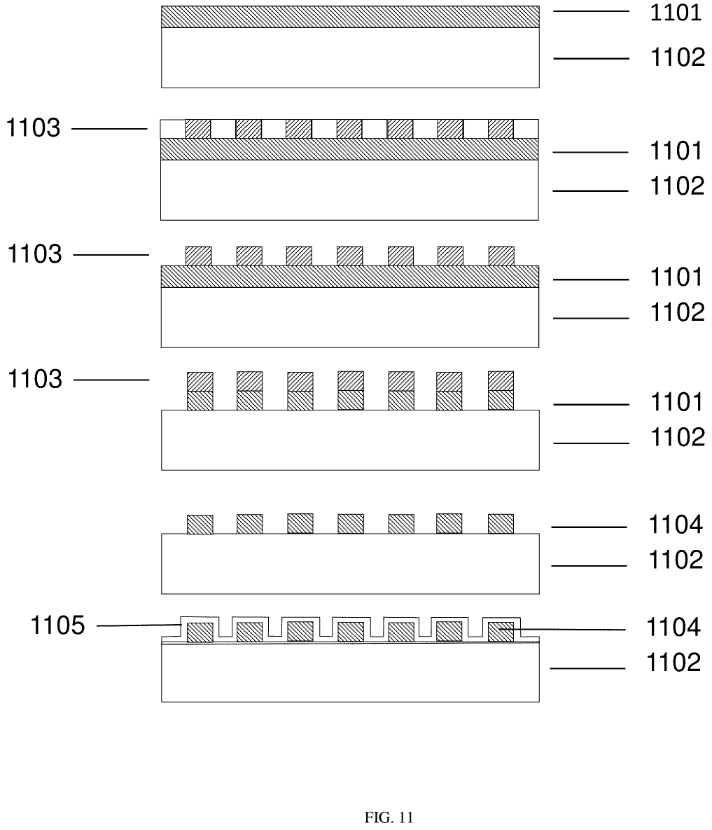

Another desirable structure comprises a rutile TiO₂ phase contained within an anatase TiO₂ phase. FIG. 11 shows a process flow for fabrication of rutile nano-islands in anatase film matrix. In this example, a rutile phase may be formed by annealing a Ti containing substrate to form rutile particles. The substrate thus modified may then be coated with a layer of TiO₂ deposited in the anatase phase or as an amorphous film that is post annealed at a temperature to form anatase, generally, a lower temperature than that for rutile formation. Alternately, an amorphously deposited film post-annealed to convert it to rutile or deposited film with the rutile crystal structure may also be used as a base layer 1101 on a substrate 1102 (FIG. 11). A mask 1103 may then be deposited on the rutile base layer 1101 and a portion of the mask removed. The mask may be a block copolymer that does not need to be patterned using photolithography, such as acrylonitrile butadiene styrene, styrene butadiene, styrene-isoprene-styrene, or ethylene-vinyl acetate. An etchant is used to remove the portions of the rutile layer that are not covered by the remaining mask using either a wet etchant or dry etchant as described elsewhere herein. After patterning the rutile layer, the mask is removed, leaving the patterned rutile surface. Another TiO₂ layer is now deposited over the rutile features. This layer may be deposited as an anatase layer or as an amorphous layer that is post-annealed to form anatase. The anatase layer may be deposited by ALD, CVD, or sputtering as described elsewhere herein. A preferred method is to use ALD to deposit a crystalline phase of anatase that is conformal over the TiO₂ layer. CVD and sputtering also afford varying degrees of conformality. The thickness of the rutile layer may vary from 1-30 nm and the lateral dimensions of the rutile features after patterning may vary from 1-50 nm, measured as a diameter or as a width of a non-circular feature (viewed from the top down). The thickness of the anatase layer may vary from 1-100 nm. Preferably, the anatase layer is from 10-40 nm. The degree of conformality, as measured by the thickness over the top of the feature to the thickness of the base of the features (between adjacent features) may be from 50-100%, preferably from 80-100% and most preferably.

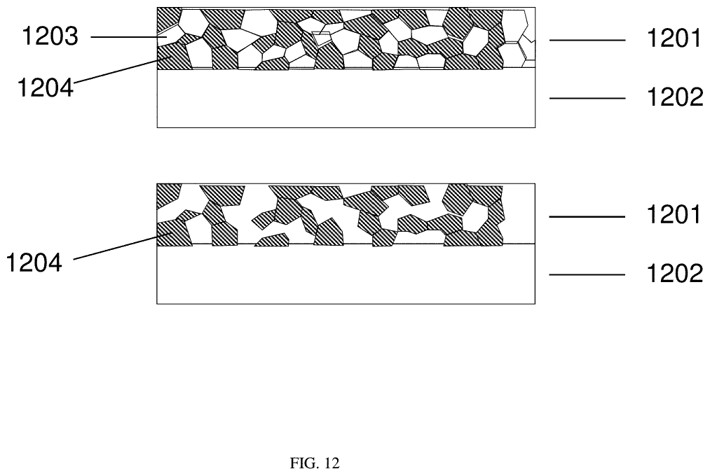

Porous structures offer the opportunity to incorporate antimicrobial moieties into a surface, and in the case or the subject invention these approaches would be a complementary technology that may work together with the photocatalytic induced antimicrobial effects. FIG. 12 shows a flow for fabrication of porous structures to contain antimicrobial materials. In this example, porous structures may be fabricated by depositing a mixture of materials and then selectively removing one. To create a nanoscale mixture using ALD, two metalorganic precursors may be co-injected to deposit a mixed layer 1201 on a substrate 1202, Figure X. The mixed layer can comprise two components, e.g., SiO₂ 1203 and TiO₂ 1204. For example, TiCl₄ and SiCl₄ may be simultaneously injected during the precursor dose and simultaneously oxidized using a suitable oxidant such as water, ozone, or oxygen plasma. The TiCl₄ and SiCl₄ may be pre-mixed in a desired ratio and delivered to a hot zone for vaporization or may be fed from separate vessels in the gas phase into the deposition chamber. Deposition temperatures are in the range of 150-300° C., more preferably 170-250° C. Pressures may be in the range of 0.1-10 Torr, more preferably in the range of 0.5-2 Torr. A number of individual layers are built up to the desired thickness, which may be between 1 and 200 nm, more preferably between 10 and 30 nm. The SiO₂ is then removed from the mixed layer by a buffered HF solution, leaving a porous structure 1204 of TiO₂. The etch rate ratio of SiO₂ to TiO₂ is high, with reported values of 490:1. The TiO₂ may be crystalline in the as-deposited condition or may be post-annealed to create the desired crystalline phase. Annealing temperatures may be between 300-600° C. Alternatively, another precursor combination may be used, for example amides with the same ligands. Other specific combinations of suitable precursors include, but are not limited to tetrakisdimethylamido titanium (TDMAT) with tetrakisdimethylamido silicon (TDMAS), tetrkisethoxy silicon [Si(OEt)₄] with tetrakisethoxy titanium [Ti(OEt)₄], and tetrkismethoxy silicon [Si(OMe)₄] with tetrakismethoxy titanium [Ti(OMe)₄]. Deposition temperatures are in the range of 150-300° C., more preferably 170-250° C. Pressures may be in the range of 0.1-10 Torr, more preferably in the range of 0.5-2 Torr. The ratio of sacrificial phase (Si) to the remaining structural phase (Ti) (i.e., the porosity) may be adjusted by the ratio of Si to Ti in the precursor mix and the pore size may be adjusted by ALD recipe, e.g., dose and purge times, temperature, etc. The porosity may be adjusted between 1-95%, more preferably between 10-50%. The overall thickness of the layer may be from 10-100 nm, more preferably from 10-30 nm. The pore size may vary from 1-10 nm.

Chemical vapor deposition may also be used to deposit the mixed film using chemically compatible precursors, e.g. TDMAT with TDMAS, Si(OEt)₄ with Ti(OEt)₄, and Si(OMe)₄ with Ti(OMe)₄. Reactive co-sputtering may also be used to form a mixed film from a mixed metal or segmented target, or by sputtering from a mixed oxide target.

Alternatively, a polymer —TiO₂ film may be formed by atomic layer deposition and the polymer removed by oxygen ashing. Suitable precursors for Ti include TDMAT, Ti(OEt)₄, Ti(OMe)₄, Ti(OiPr)₄, and Ti(thd)₂(OiPr)₂, where thd=tetramethaneheptanedianoto and OiPr=isopropoxide. Suitable polymer precursors include ethylene glycol, polyols, or an amine, such as ethanolamine. Deposition temperatures are in the range of 150-250° C.

Another alternative is to leave the polymer in place and use it to absorb a therapeutic material, e.g., an antibiotic, that may elute over time when placed in the body, thus providing an extended antimicrobial action.

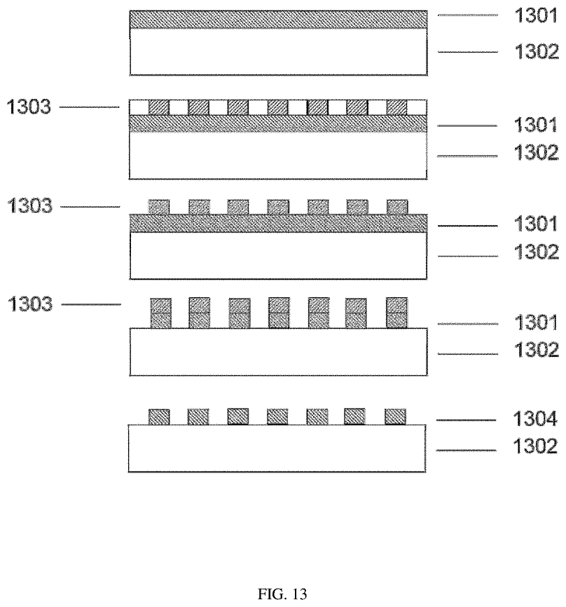

As described above, it may be useful to introduce topographic features in the modified surface using additive or subtractive methodologies. FIG. 13 shows a process flow for fabrication of topographic structures. Nanoscale features can be produced by a number of methods, including additive and subtractive approaches, or combinations thereof. Desirable nanoscale features include TiO₂ based regions, or nodules that protrude from the surface. Nanoscale porous features are also desirable.

In order to produce free standing TiO₂ nanoscale features that are well adhered to the surface, the desired TiO₂ film may first be deposited. It may have the desired crystal structure in the as-deposited state or may be thermally treated (post-annealed) after deposition to produce the desired phase. Anatase is one desirable phase for photocatalytic properties that can be antimicrobial. FIG. 13 shows a TiO₂ film 1301 deposited on a substrate 1302. The substrate may be planar, as shown in the figure, or have a curvature or even a 3-dimensional nature as described earlier, e.g. it may be filamentary or fibrous. A mask may be applied to the surface, e.g., a block copolymer 1303, that is then etched to remove one of the block components. The exposed TiO₂ may then be etched by suitable means, for example a wet or dry (vapor) chemical. Etchants useful for TiO₂ include hot sulfuric acid, hot phosphoric acid, and hot phosphoric acid/hydrogen peroxide mixtures. Examples dry etchants for TiO₂ include CF₄/Ar/O₂ mixtures. The remaining mask 1303 is then removed to expose isolated TiO₂ features 1304. The overall thickness of the free standing TiO₂ may be from 1-100 nm, more preferably from 10-30 nm. The size of the TiO₂ particles measured within the plane of the film may be between 2-30 nm depending on the nature of the block co-polymer mask, i.e., the size and spacing of its constituents.

Turning now to the coating, it is comprised of titanium dioxide that is deposited by atomic layer deposition (ALD). ALD is a layer by layer growth process that produces highly conformal coatings with thickness control at the atomic scale. The process is generally carried out in vacuum wherein alternating doses of a precursor containing the metal cation and an oxidizer are applied to the workpiece at elevated temperature. The precursor dose and oxidizer dose are separated by an inert purge. Suitable precursors for TiO₂ include Ti halides, alkoxides, amides, amidinates, guanidantes, and cyclic hydrocarbons, e.g., cyclopentadienyls. Preferred precursors for TiO₂ include tetrakis dimethylamido titanium (TDMAT) and titanium tetrachloride. Precursors may be delivered to the deposition chamber via a carrier gas flowing through a heated vessel. Alternatively, the precursor may be dissolved in a solvent and delivered to a vaporization zone where it is mixed with a carrier gas for transport to the deposition chamber. Suitable oxidizers include water, ozone, and oxygen plasma. Suitable inert purges include nitrogen and argon. Solvents that may be used for liquid delivery of the precursor include linear and cyclic alkanes and alkynes. Suitable carrier gases include nitrogen, argon, and helium. Dose and purge times are sufficiently long to allow diffusion of the species into the recesses of the device, which may range from sub-second to minutes. Deposition temperatures are in the range of 150-250° C.

The ALD process and related processes may be tuned to provide a multiphase constitution for the photocatalytic film which further provides photocatalytic response to visible wavelengths of light. For example, the film may comprise a mixture of two or more of the following phases: anatase, rutile, or brookite. While the anatase phase is generally regarded as the most photoactive phase, other phases, particularly rutile, are able to inject photostimulated charge into the anatase regions, thus imparting visible light activity. The deposition process may comprise multiple steps with the first portion of the film deposited under one condition and another portion of the film deposited under a different condition. For example the first portion of the film is deposited at a lower temperature in the range given above and the second portion of the film is deposited at a higher temperature in the ALD range. The film may also be deposited in two steps with an annealing step performed between the two steps and optionally after the second deposition.

Doping may also be employed to shift the frequency of the photocatalytic response to visible wavelengths. In particular, cerium may be added to the TiO₂ film. The cerium oxide may be added as a discrete series of layers that are adjacent to each other, or interspersed with TiO₂ layers. The CeO₂ layer may be located below the surface of the TiO₂ film. The TiO₂ and CeO₂ may also be simultaneously deposited as a mixture using precursors for each metal that are compatible in the gas and liquid phase. Suitable precursors for CeO₂ include Ce halides, alkoxides, amides, amidinates, guanidantes, and cyclic hydrocarbons, e.g., cyclopentadienyls. Preferred precursors for CeO₂ include trisisopropylcyclopentadienyl cerium and mixed ligand precursors comprising cyclopentatdienyls and amidinates. Similar Ti precursors are used for simultaneous deposition of CeO₂ and TiO₂. Precursors may be delivered to the deposition chamber via a carrier gas flowing through a heated vessel. Alternatively, the precursor may be dissolved in a solvent and delivered to a vaporization zone where it is mixed with a carrier gas for transport to the deposition chamber. Suitable oxidizers include water, ozone, and oxygen plasma. Suitable inert purges include nitrogen and argon. Solvents that may be used for liquid delivery of the precursor include linear and cyclic alkanes and alkynes. Suitable carrier gases include nitrogen, argon, and helium. Dose and purge times are sufficiently long to allow diffusion of the species into the recesses of the device, which may range from sub-second to minutes. Deposition temperatures are in the range of 150-300° C. The deposition temperatures in this range are compatible with stainless steel, titanium, titanium alloys, and ZrO₂—Al₂O₃ ceramics. Deposition temperatures may also range from 150-250° C. This range is compatible with PEEK.

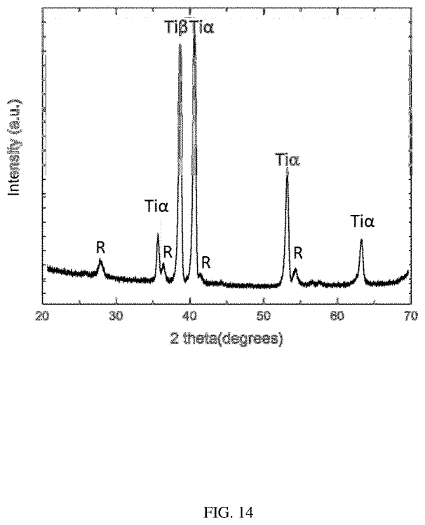

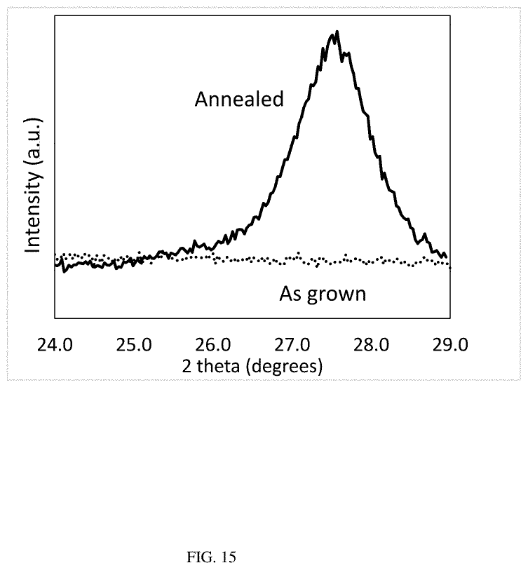

Several of the preceding examples describe the utility of a rutile layer and the corresponding rutile-anatase interface. Rutile may be formed by annealing titanium or titanium containing alloy substrates, in the temperature range of 350-750° C. with a heating and cooling ramp range of 2-10° C. per minute. The substrate can be at the annealing temperature for 0.1-6 hours. The annealing process can be performed in air, nitrogen, or a mixture of nitrogen and oxygen. Prior to annealing, the substrate can be cleaned using multiple steps. Each step can take 5 min to 2 hours. In one step the substrate can be rinsed using ultrasonic bath at the temperature of 25-100° C. The cleaning agent can be a neutral cleaner, DI water, alcohol or acetone. The cleaning can also be done by merging the substrate in an acid or a base bath. FIG. 14 shows a grazing incidence x-ray diffraction (XRD) pattern of a Ti-6Al-4V substrate annealed in a nitrogen/oxygen mixture, resulting in a phase pure rutile film of approximately 30 nm thickness. Rutile diffraction peaks are indicated by R. Titanium diffraction peaks are indicated by Ti_{β}, Ti_{α}. Rutile may also be formed by post processing of an amorphous film formed by atomic layer deposition or chemical vapor deposition. FIG. 15 shows an XRD pattern for an approximately 30 nm film grown by ALD with an amorphous phase and then converted to rutile crystal phase by annealing at a temperature in the range of 350-650° C. for 1-3 hours. In another example a titanium or titanium containing alloy can be first cleaned and then annealed at a temperature between 350-550° C. to form an anatase substrate surface.

Combinations of rutile and anatase layers can thus be formed, for application to the layer structure principles described above. In another example, an amorphous film grown by ALD on anatase or rutile substrate surface can be annealed at the temperature between 300-500° C. to form an anatase film.

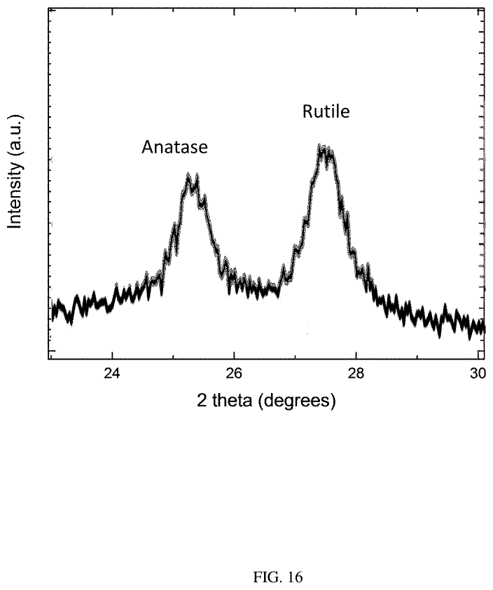

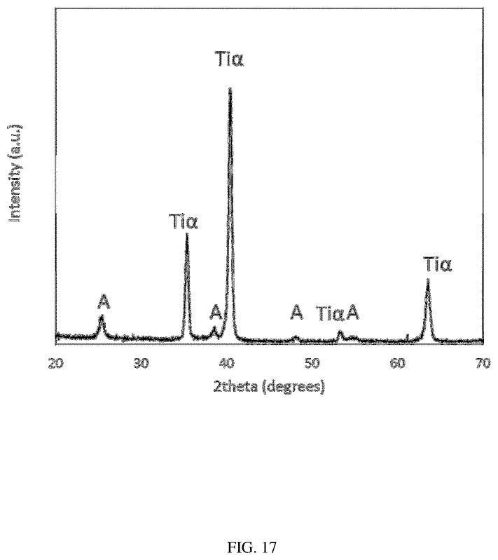

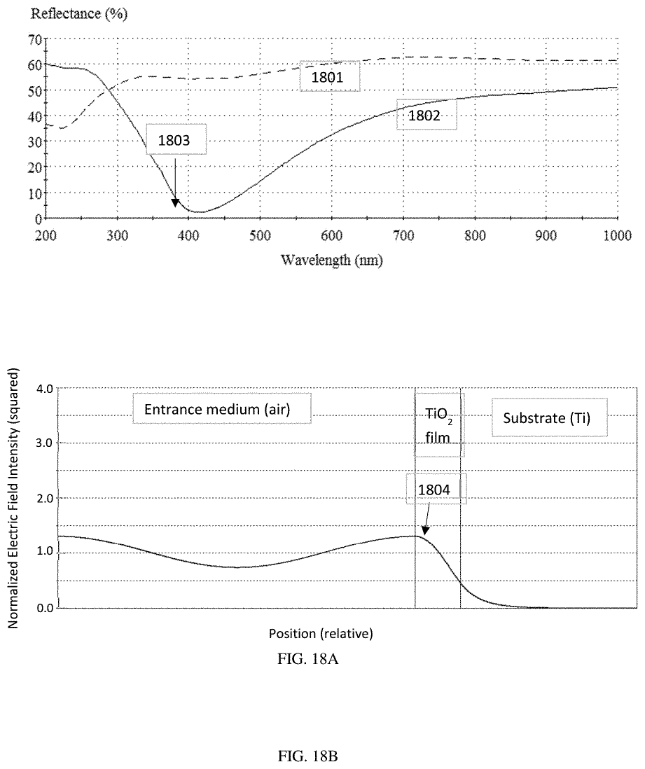

In one example related to a several previous examples, the outer anatase layer may be formed in-situ on a Ti-6Al-4V substrate, and on other surfaces as well, including on alternative substrate materials, titanium native or thermal oxides, rutile under layers or stainless steel. In these cases there is no post annealing needed to form the anatase layer from a precursor layer, and there are significant advantages to this in terms of an efficient fabrication process flow. FIG. 16 shows an XRD pattern for an anatase as grown film on a rutile crystal phase underlayer. FIG. 17 shows an x-ray diffraction pattern for an anatase film (as-deposited) on a Ti-6Al-4V substrate.

A key aspect of the subject invention is the creation of layer structures (“coating”) to comprise surgical invasive modified surfaces. There are several design principles that are presented herein.
a) Formation of an anatase layer on the outer surface to maximize the hydroxyl radical surface density and hence the photocatalytic effect
b) Conformal formation of layers and layer systems, to replicate substrate surface topography and to increase topography via crystallization and growth phenomena in the layers.
c) Introduction of under layers such as rutile, CeO₂ and CeO₂ rutile, and CeO₂ nanoscale islands in anatase, to increase optical absorbance and to act as charge injection sources into the outer anatase layer
d) Choice of optimal layer thicknesses and compositions in order to maximize optical absorption at a photocatalytic illumination wavelength.
e) Choice of optimal layer thicknesses and compositions in order to maximize the optical reflectance at a photocatalytic illumination wavelength.

Photocatalytic materials employ optical illumination to separate charges in a semiconductor such as TiO₂. For bulk photocatalytic ceramics or powders or thick layers (e.g. greater than 250 nm thickness), there may be ample ability to couple light into the material. For thin films, e.g. less than 250 nm thickness, and especially so for less than 100 nm thickness, it is particularly important to efficiently couple the light into the material. Several methods to achieve this are discussed above, including use of a rutile layer, which has increased optical absorption relative to anatase, or by incorporating rutile nanocrystals in an anatase layer, or incorporation of CeO₂ with anatase, either in a homogenous CeO₂—TiO₂ solid solution, or as a discrete charge injection layer.

All of these aspects of the subject invention utilize thin film layers with thicknesses on the order of photocatalytic illumination wavelengths, and that are transparent at those wavelengths. For practical photocatalytic antimicrobial devices in the subject invention, preferred illumination wavelengths are in the 365-430 nm spectral region, more preferably in the 385-410 nm region.

Hence optical interference effects are valuable tools to identify preferred embodiments of these single layer and multilayer film thicknesses. These effects result from reflections from each interface between layers, and the multiple reflections interfere with each other, either constructively or destructively, and those affects have a high degree of wavelength dependence. In these applications a layer's optical thickness is the critical parameter, at a wavelength of interest, such as for photocatalysis. Optical thickness of a layer is the product of its physical thickness and its refractive index (at a wavelength). Optical interference takes place and is generally useful for transparent or semitransparent layers that have optical thickness ranging from 0.05 to 5 times the illumination wavelength. A variety of device optical properties may be achieved using single or multiple layers of transparent materials. These include antireflection coatings, high reflectors, bandpass filters, short wavelength pass filters etc.

Optical thin film computational models are routinely used for design of optical coatings, using commercially available software, and based on known optical constants for the constituent materials. The exact values for optical constants may vary for a material, depending on how it is made, density, crystal phases present, etc. Use of optical thickness as a design parameter essentially compensates for those variations.

The subject invention includes use of these principles to improve the performance of photocatalytic layers in the present invention. From this optical standpoint, there are two considerations in determining the target thicknesses for such layers that comprise the invasive devices modified surfaces:
1) Maximizing optical absorbance in specific regions in the film layer system,
2) Maximizing reflectance in order to propagate photocatalytic illumination into deep micrometer scale porous structures.

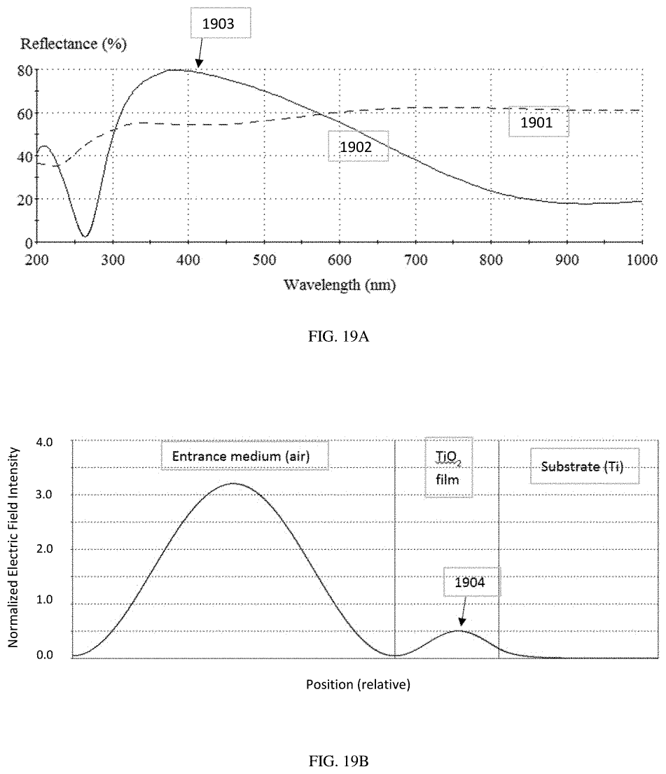

Realizing these effects via layer thicknesses and compositions are an important design principal to optimize photocatalytic devices that employ thin layers. One example is for a single photocatalytic layer of TiO₂ on a chemically pure titanium substrate. For an illumination wavelength of 405 nm incident on a titanium substrate, an antireflection coating can be formed, at that wavelength and at normal incidence, using a single TiO₂ layer. FIG. 19a shows optical model results for spectral reflectance for a 26.5 nm TiO₂ film on an opaque titanium metallic substrate 1902. It has a single reflectance minimum of about 2% at the 405 nm design wavelength 1903. Spectral reflectance of the bare (uncoated) titanium 1901 is also shown for comparison.

The optical thickness of this layer is 67.3 nm, calculated as the product of the refractive index at that wavelength (n=2.54), and thickness (t=26.5 nm). Optical thickness may also be expressed as the number of “full waves” at the design wavelength, in this case, 405 nm. The number of full waves for a transparent or semi-transparent layer is defined as the optical thickness divided by the wavelength, and is unit-less. Thus such a single layer antireflection (AR) coating using TiO₂ on this substrate is 0.166 full waves optical thickness, at 405 nm. For this type of optical coating design, with fairly broad spectral features, the tolerance for this optical thickness is somewhat relaxed, e.g. ±10%. This is a general rule for antireflection coatings on titanium that is independent of the illumination wavelength, at least in spectral ranges where the titanium optical constants do not vary greatly, as for the mid-visible through the UVA region (365-550 nm). Applying this principle to another possible photocatalytic illumination wavelength, an AR coating for titanium at 365 nm illumination is also 0.166 full waves (60.6 nm optical thickness due to TiO₂ dispersion), or 23.2 nm physical thickness.

Antireflection properties may not have specific or direct utility for the subject photocatalytic devices, but these and other optical interference designs offer a way to concentrate phototcatalytic illumination in specific regions in the layer or layers systems, which has utility to increase the photocatalytic antimicrobial response for a given illumination intensity. For a given wavelength and incident angle, these interference effects create standing optical waves in the layers, entrance medium (usually air) and in the substrate, if it is transparent. In the case of opaque substrates, e.g. metal or semiconductor substrates above the bandgap, these fields are attenuated and disappear at greater depths. These optical waves are essentially electric fields (E-fields), forming a standing optical electric field, and there are varying E-field strengths, including nodes and antinodes throughout the layers, and are calculated and plotted as E-field squared vs position normal to the surface. E-field squared is proportional to intensity. FIG. 19b shows E-field distribution (illumination at 405 nm, normal incidence) in the single layer TiO₂ on titanium described above. For this design there is a peak in intensity at the outer part of the TiO₂ layer 1904, i.e. at the outer surface where photo-generated electrons and holes will be employed to generate ROS, and the photocatalytic effect may be increased in this example.

The layer thicknesses and compositions can thus be used to engineer high input optical power at specific parts of the layers, and there are a wide range of photocatalytic and photometric performance criteria, and corresponding layer design options that may be beneficially devised using these principles.

In other examples and layer configurations described herein, high E-fields may be aligned with rutile under layers, with regions that have nano-islands of platinum or rutile embedded in an anatase matrix, or for underlying CeO₂ layers.

A related embodiment is the creation of a high reflectance properties. In some substrate surfaces that have somewhat large features and deep porosity, the wavelength of photocatalytic illumination may decrease in intensity for regions that are deeper in the structure. In cases where the porosity is large relative to the wavelength, (e.g. greater than 20× the wavelength, geometric optics effects can be employed. Thus, increases in photocatalytic illumination reflectance at the pore surfaces will allow light to reach deeper into the pores, and thereby create increased antimicrobial efficacy in those other wise shadowed regions.

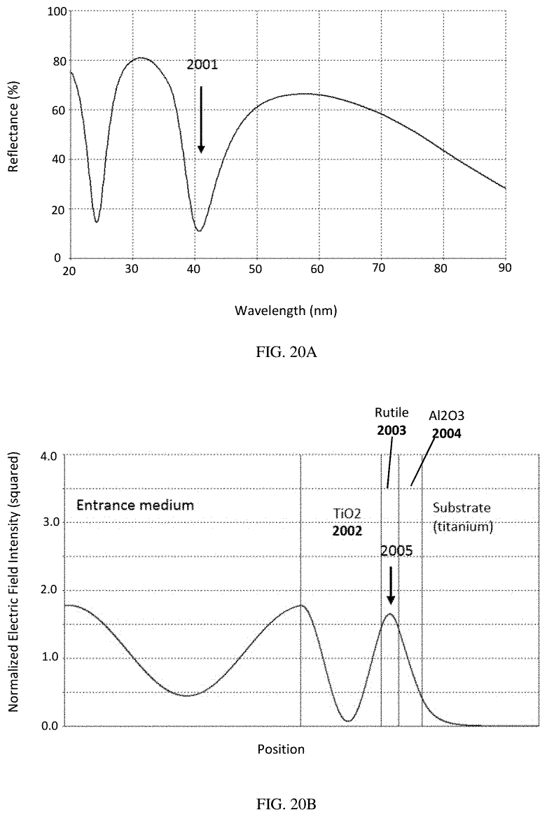

For titanium and titanium alloys, reflectance is rather low at around 55% in the UVA (365-400 nm), because these metals do not have the density of free electrons that are seen in high reflectance metals such as aluminum and silver. FIG. 20a shows optical model results for spectral reflectance of the bare (uncoated) titanium 2001. Spectral reflectance 2002 for a 65.2 nm TiO₂ film on an opaque titanium metallic substrate is also shown, at its peak, at 390 nm wavelength, it has approximately 80% reflectance 2003. This increase in reflectance is due to the single layer of TiO₂, resulting from its optical interference effects with the titanium substrate.

In general there are more sophisticated ways to further increase reflectance, such as by incorporating alternating layers of low and high index materials, known as a dielectric reflector. In principle a dielectric reflector could be formed from alternating layers of anatase and rutile, for example. For photocatalytic applications, anatase TiO₂ and aluminum oxide are reasonable choices for other interference coating designs such as those disclosed herein, because of their refractive index difference and their physical stability, chemical stability and biocompatibility.

An enhanced high reflector utilizes a reflective metal such as platinum or aluminum, with a dielectric reflector fabricated on top of it. Any of these approaches are suitable to incorporate various forms of TiO₂, especially anatase, layers on the outer surface of a coating, as provided in the subject invention.

For the case of the simple reflector of FIG. 20, the E-field distribution (FIG. 20b) has a different nature, and the E-field within the 62.5 nm thickness TiO₂ layer is somewhat lower than in the case of the antireflection coating, and the peak is located near the center of the layer 2004. In that case the optical thickness of the TiO₂ is 161.6 nm (based on n=2.585 at 390 nm wavelength and t=62.5 nm), or 0.42 waves optical thickness at 390 nm. This single TiO₂ layer optical thickness of 0.42±0.04 full waves, is a design principle at any illumination wavelength in the 360-430 nm wavelength range, to achieve 80% reflectance on titanium or titanium alloy substrates at the photocatalytic illumination wavelength.

Thus, in these simple examples, employing a single layer of TiO₂ on titanium, there is a tradeoff between E-field amplitude, E-field location, and reflectance at the photocatalytic illumination wavelength. The choices in this tradeoff may be influenced by the particular invasive device hardware, surface topography, and other system and methodology aspects. Surfaces with deep features may benefit from higher reflectance, and surfaces with smooth surfaces and no deep features may benefit from optimization of the illumination E-field at the outer surface of an anatase layer.

There are several approaches to address these performance criteria. For a high reflectance configuration of FIG. 20, rutile, CeO₂ or other charge injector moieties discussed above could be located at the antinode (maximum) of the E-field amplitude near 2004. Other dielectrics may also be judiciously added to position E-fields (and increase absorbance) while maintaining reasonable reflectance levels, e.g. greater than 60%.

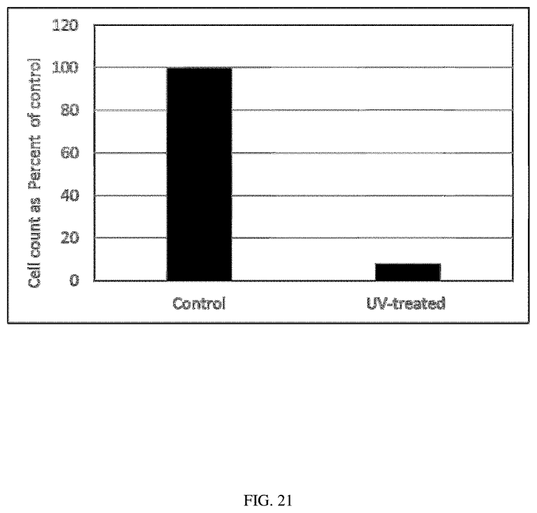

FIG. 21 shows spectral reflectance (FIG. 21a) and E-field distribution (FIG. 21b) for a 3 layer coating design on a titanium substrate. The layers are: a 69 nm TiO₂ anatase layer 2102 on the outside (adjacent to the entrance medium air), a 15 nm rutile layer 2103, and a 20 nm Al₂O₃ layer 2104 adjacent to the substrate. It can be seen that there is still a reflectance minimum 2101 at the photocatalytic illumination wavelength 405 nm, although significantly higher reflectance than in previous examples. The cogent aspect is a strong E-field amplitude maximum 2105 in the 15 nm rutile layer, which will increase coating absorbance and therefore the photocatalytic antimicrobial properties of the device. Other materials could be aligned with high E-fields using these principles, including other photocatalytic compounds, metal nano-islands such as the platinum group metals.

FIG. 21 shows bactericidal data for an approximately 30 nm TiO₂ film in the as-deposited anatase phase on a silicon substrate. The film was exposed to 2E+7 colony forming units (CFU)/ml of Staphylococcus aureus and then irradiated at 365 nm for 3 hours with the LED light power of 1.6 mW/cm² at the sample surface. A reduction of 90% of the bacteria were observed relative to a control sample without coating (bare glass) and no illumination. Further, TiO2 coated samples were autoclaved after initial testing and showed similar or improved antimicrobial performance under illumination. Performance improvements were between 5-10%.

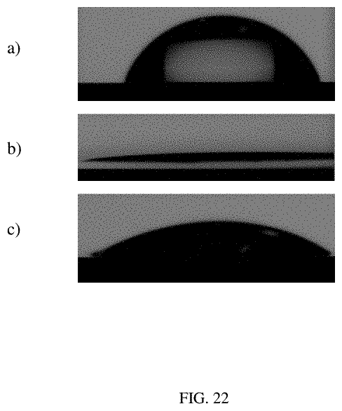

FIG. 22 shows a contact angle image before and after UV irradiation. Contact angle images of a TiO₂ film on a silicon substrate, irradiated at 365 nm for 300 minutes. a) before, b) immediately after and c) 3 days after UV irradiation. Super hydrophilicity (contact angle ˜0 degrees within 3 seconds of placing a water drop on the film) was photo-induced in the film and then decayed but remained hydrophilic over a period of 3 days after the irradiation with a final contact angle of 17 degrees. While not being bound by theory, the development of a highly hydrophilic TiO₂ surface is thought to be due to the preferential absorption of water on the photogenerated surface defective sites. The electron-hole pairs generated by the light accumulate at the conduction band and the valence band. Electrons will be transferred to oxygen adsorbed on the surface, while holes at the valence band create oxygen vacancies. The oxygen vacancy sites are favorable for dissociative water adsorption and promoting hydrophilicity. Implantation in the body removes the direct illumination of the surface. However, the contact angle measurements show that the TiO₂ films is expected to only slightly increase in contact angle of water (indicating the depletion of the photogenerated surface defective sites) but remain hydrophilic. The hydrophilicity of the surface can further increase the antimicrobial effect by preventing the bacteria from adhering to the surface and promoting osseointegration by increasing the surface energy.

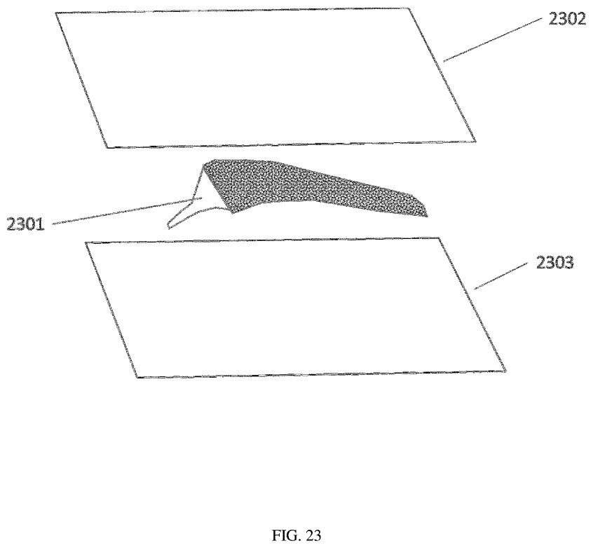

FIG. 23 shows an exploded schematic of an orthopedic invasive device 2301 in transparent packaging that may be illuminated prior to its use. A portion, for example the top 2302, or all of the package including the top 2302 and bottom 2303 are transparent to illumination of the desired wavelength of light. Wavelengths in the range of 365 nm-410 nm may be used, preferably in the range of 385 nm-410 nm, most preferably in the range of 405-410 nm. The transparent portion is a polymeric material, for example low density polyethylene, polyester, polyethylene copolymer, ethylene vinyl acetate, polypropylene, and polystyrene, or laminates thereof, for example polyethylene-cellophane, polypropylene-cellophane-polyethylene, or polycarbonate-polyethylene. One side of the package, for example, the bottom 2303 may optionally be reflective so that the illumination impinges on the underside of the device. Reflectors include mylar films, or a metallized coating on a polymer, for example, aluminum. The package, including adhesives that may be used to bond the edges, is compatible with gamma ray sterilization.

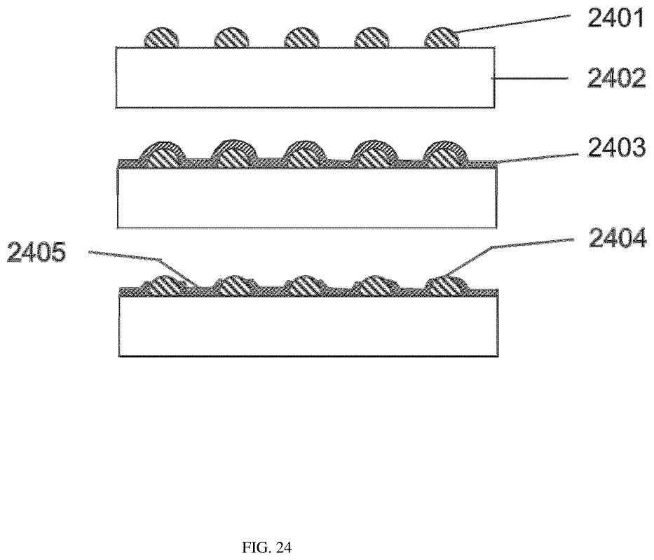

For certain applications, e.g., surgical instruments, it may be desirable to enhance the wear resistance of the coating. The instrument may be made of a Ti alloy or stainless steel. A wear resistant coating may be fabricated by applying the coating over hard particles adhered to the surface of the surgical instrument. Fine, hard particles may be any hard material, but preferably aluminum oxide (MOHS hardness 9). The particles may be deposited by a vapor means such as sputtering (optionally in an oxygen ambient), evaporation, ion beam deposition, CVD, or ALD. In the case of ALD, underdosing may be used to create an island-like structure. Suitable ALD precursors include tetramethyl aluminum (TMA). Suitable oxidizers for ALD with TMA include water, ozone, and oxygen plasma. An example surface modification is shown in FIG. 24. The particles 2401 thus deposited on the substrate 2402 are then overcoated with a TiO₂ layer 2403 using any of the preferred methods described herein. If some of the relatively softer coating (TiO₂ MOHS hardness=6.2) at the tips of the surface asperities 2404 wear, the hard particles forming the asperities prevent further wear of the TiO₂ layer 2405. Thus, an antimicrobial behavior of the surface is preserved by virtue of the remaining TiO₂ in between the hard particles 2401. The desired range of particle size is 10-10000 nm, more preferably 50-500 nm. The coating thickness of photocatalytic TiO₂ may be 10-100 nm, more preferably 20-50 nm. An aspect ratio of the coating thickness measured from an unworn location to the particle size may be from 1-100, more preferably from 1-10. The coating may comprise multiple layers of different phases of rutile and anatase as described elsewhere herein. The individual layers may be in the range of 5-20 nm. The thickness of the coating at a location between particles and the top of the particles may be between 80-100%, or more preferably 90-100%. The thickness of the coating at a location proximate to the base of a hard particle in the radial direction of a hard particle may be within 80% of a thickness proximate to the base of the film in the direction of the substrate. Preferably this ratio is 85%, and more preferably 90%. Other materials may be used for the hard particles, e.g., SiC (MOHS hardness 9-9.5) or BN (MOHS hardness 9.5). Desirable ranges of the ratio of the MOHS hardness of the hard particles to the coating are in the range of 1.45-1.53.

The subject invention may be embodied in the forgoing examples and embodiments that are by no means restrictive, but intended to illustrate the invention. Different embodiments and examples given previously may be freely combined.

What is claimed is:

1. A surgical invasive hardware device with a modified surface comprising; a surgical invasive device, the surface of the device having a coating of material comprising TiO₂, the coating having a total thickness between 1-80 nm, the coating having at least 80% conformality on the surface of the device, the coating further having photocatalytic bactericidal properties induced by light exposure.

2. The device of claim 1 where the device is comprised of titanium or a titanium alloy.

3. The device of claim 1 where the device is comprised of a stainless steel alloy.

4. The device of claim 1 where the device is comprised of PEEK.

5. The device of claim 1 where the device is a joint implant.

6. The device of claim 1 where the device is an external fixating pin.

7. The device of claim 1 where the TiO₂ coating comprises an anatase layer.

8. The device of claim 1 where the TiO₂ coating comprises an anatase layer and a rutile layer.

9. The device of claim 1 where the TiO₂ coating comprises rutile particles covered by an anatase TiO₂ layer.

10. The device of claim 1 where the photocatalytic bactericidal property is induced by light with a wavelength ≥365 nm.

11. The device of claim 1 where the photocatalytic bactericidal property is induced by light with a wavelength ≥385 nm.

12. The device of claim 1 where the photocatalytic bactericidal property is induced by light with a wavelength ≥405 nm.

13. The device of claim 1 where the coating comprises 0.1-5% CeO₂.

14. The device of claim 1 where the coating comprises discrete layers of CeO₂ separated by at least one layer of another oxide material

15. The device of claim 1, wherein the coating further comprises platinum, iridium, rhodium or palladium metal particles of 2-20 nm diameter.

16. A surgical invasive hardware device with a modified surface comprising; a surgical invasive device, the surface of the device having a deposited coating of material, wherein the coating thickness and composition increase standing optical electric fields at a photocatalytic illumination wavelength in certain parts of the coating.

17. The device of claim 16, wherein the maximum amplitude of the standing optical electric field is at the outer surface of the deposited photocatalytic coating and the amplitude is reduced in a direction downward into a portion of the coating.

18. The device of claim 16 wherein the coating comprises at least two layers.

19. A surgical invasive hardware device with a modified surface comprising; a surgical invasive device, the surface of the device having a coating of material comprising TiO₂, the coating having a total thickness between 1-80 nm, the coating having at least 80% conformality on the surface of the device, the coating having photocatalytic bactericidal properties induced by light exposure, the coating further incorporating wear resistant particles, the wear resistant particles being adhered to the device surface.

20. The device of claim 19 wherein the wear resistant particles are Al₂O₃ or SiC.
